(19) United States
(12) Patent Application Publication
(10) Pub. No.: US 2004/0039359 A1
(43) Pub. Date: Feb. 26, 2004

(54) Disposable menstrual undergarment

(76) Inventors: Leigh M. Krieps, Knoxville, TN (US); Manya H. Pirkle, Knoxville, TN (US)

Correspondence Address: PITTS AND BRITTIAN P C, P O BOX 51295, KNOXVILLE, TN, 37950-1295, US

(21) Appl. No.: 10/645,417

(22) Filed: Aug. 21, 2003

Related U.S. Application Data
(63) Continuation of application No. 10/145,997, filed on May 15, 2002.
Application No. 10/145,997 is a continuation-in-part of application No. 09/895,750, filed on Jun. 29, 2001, now abandoned.

(51) Int. Cl. A61F 13/15; A61F 13/20

(52) U.S. Cl. 604/358

(57) ABSTRACT

A disposable menstrual undergarment configured to be worn as a panty for absorption of menstrual fluids. The panty includes an exterior layer of breathable non-porous material, a middle layer of absorbent materials, and a removable interior layer of absorbent materials positioned on the middle layer and within the front portion, rear portion, and the crotch portion of the undergarment. At least one strip of absorbent materials is positioned along each interior portion of each leg opening. The disposable menstrual undergarment provides for absorption of fluids for young females and/or for adults needing special assistance, with an interior layer that is removable after becoming soiled, while providing additional layers of absorbent materials within the undergarment for absorption of fluids for an extended time until a replacement undergarment is obtained.

### CROSS-REFERENCE TO RELATED APPLICATIONS

This is a continuation-in-part of application Ser. No. 09/895,750, filed Jun. 29, 2001, now abandoned.

### STATEMENT REGARDING FEDERALLY SPONSORED RESEARCH OR DEVELOPMENT

Not Applicable.

### BACKGROUND OF THE INVENTION

1. Field of Invention

This invention relates to a disposable menstrual undergarment. More specifically, the present invention relates to a disposable menstrual undergarment for wear by young females, or for wear by adults needing special assistance.

2. Description of the Related Art

The prior devices for protecting young females who begin menstruation typically include a disposable panty containing thick absorbent materials lining the central portion of the panty along an inner crotch area of the panty. Alternatively, a typical panty for menstrual wear may have an elongated depression along an inner crotch area of the panty for placement of a feminine napkin that is removable by the wearer. Additionally, prior absorbent pants include a menstrual short having a catamenial pad assembly connectable to the crotch area of the menstrual short, with elastic leg openings having a plurality of crimps and/or elastic ridges around each leg opening. For the prior panties, the user must remember to periodically replace the catamenial pad assembly in the crotch area, when the catamenial pad becomes soiled, for continued protection from leakage by the menstrual short. The prior diapers and panties must be properly positioned on the wearer, and must be changed periodically, to provide adequate absorption of menstrual fluid or other excreted fluids. Further, the prior diapers and panties must be periodically inspected by the wearer or a person associated with the wearer, to check for the need of replacement of the absorbent pads or liners. Teachers and/or day-care staff are naturally hesitant to take on the responsibility of reminding young girls of a pre-teen age to periodically check undergarments and/or panties for menstrual releases. Further, pre-teen girls may be shy and hesitant to request permission to visit a rest-room during class sessions in school.

For young females of the ages of 8 to 14 years and who are beginning menstruation, the young females do not normally know in advance when menstrual releases will occur. It is also typical that the young females may not remember to change absorbing pads or feminine napkins at periodic intervals for continuous protection throughout a day in school. Further, young females typically do not prefer wearing a bulky panty under their garments that may be interpreted to be a diaper undergarment.

Therefore, there is a need for a pull-on panty that is light-weight, stretchable, disposable, and includes at least one removable interior layer. Further, there is a need for a pull-on panty including removable absorbent layers on the crotch interior, and absorbent materials along each leg opening interior, for absorption of fluids to prevent leakage from the panty without the need for periodic replacement of absorbent pads.

### BRIEF SUMMARY OF THE INVENTION

In accordance with the present invention, there is provided a disposable menstrual undergarment for absorption of body fluids including a front portion, a rear portion, and a crotch portion. The undergarment includes an exterior layer of breathable non-porous material, a waistband of generally continuous material, and two leg openings adjacent the crotch portion. Each of the leg openings includes an interior faced absorbent border. The undergarment includes a middle layer of absorbent materials and an interior faced, removable layer of absorbent materials along the interior surfaces of the front portion, rear portion, and the crotch portion of the undergarment. The interior faced, removable layer of absorbent material and the interior faced absorbent border along each interior faced surface of each leg opening provide for absorption of fluids excreted by the wearer of the undergarment. The interior layer of absorbent materials is removable by the wearer from the middle layer when the interior layer is soiled, with the middle layer remaining fixed against the crotch portion and the border of absorbent materials remaining positioned along the interior faced surfaces of each leg opening. The disposable menstrual undergarment provides protection for young females and adults needing special assistance during a day of school or during day-long adult outings, while providing an undergarment that is not bulky, is easily replaceable, and is inexpensive while being fully absorbent of any fluids excreted by the wearer of the undergarment.

### BRIEF DESCRIPTION OF THE SEVERAL VIEWS OF THE DRAWINGS

The present invention is illustrated in the drawings in which:

FIG. 1 is a front view of a disposable menstrual undergarment;

FIG. 2 is a rear view of FIG. 1 of the present invention;

FIG. 3 is a plan view of the disposable menstrual undergarment laid open;

FIG. 4 is a section view 4-4 of FIG. 3, along the leg and crotch area; and

FIG. 5 is a section view 5-5 of FIG. 3, along a rear interior portion of the undergarment.

### DETAILED DESCRIPTION OF THE INVENTION

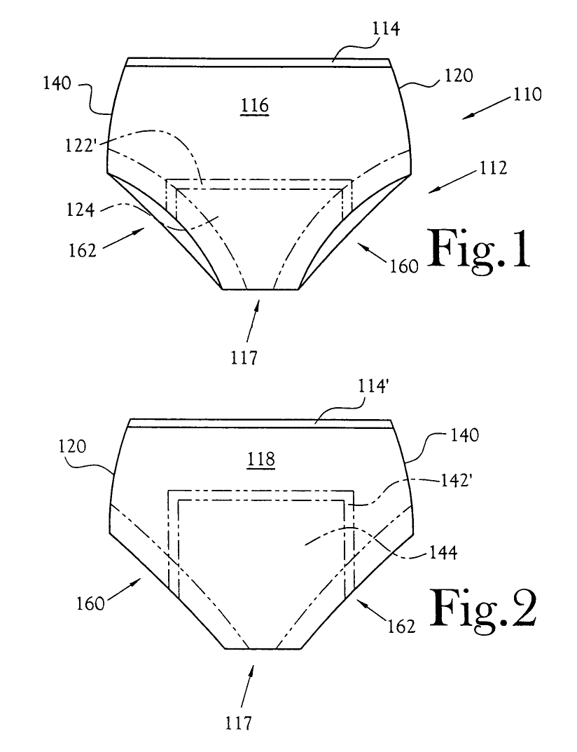

An improved disposable menstrual undergarment 110 is illustrated in FIGS. 1 and 2. The undergarment is configured in the shape of a panty 112 to be worn to enclose the wearer's abdomen, hips, buttocks, and pelvis in order to absorb, collect and contain menstrual fluids, and/or other fluids or solid materials excreted by young girls, and/or incontinent individuals. The panty 112 is intended to be discarded by the wearer after a single use, and is not intended to be laundered or cleaned for reuse after soiling of the interior of the panty 112.

As illustrated in FIGS. 1 and 2, one embodiment of the panty 112 includes an outer front portion 116, for coverage of the abdomen of the wearer, and an outer back portion 118, for coverage of the buttocks of the wearer. An outer layer of the front portion 116, a crotch portion 117, and a back portion 118 includes a material composed of cellulose and/or synthetic polymers. The panty 112 includes a generally continuous waistband 114, 114′ having an elastic material attached thereon for maintaining the panty 112 around the wearer's waist. The outer sides 120, 140 cover the respective left and right sides of the wearer's hips, and include an outer layer of material that is generally continuous. At least one connecting seam (not shown) is oriented perpendicular to the waistband 114, 114′ and along one or both outer sides 120, 140. Alternate connecting seam or seams orientation will be recognized by those skilled in the art for connecting the outer layer of material to form a generally continuous panty 112.

Each connecting seam is connected by an attaching means known to those skilled in the art, such as stitching, bonding with glue, and/or forming seams with heat and pressure, for attaching the outer sides of the front portion of the panty to the outer sides of the rear portion of the panty. The outer portions of the waistband 114, 114′ and each outer side 120, 140 of the panty 112 include a cellulose-based fibrous material that incorporates a plurality of elastic filaments interdisposed within the fibrous material, to allow outer portions of panty 112 to be stretched to a limited extent in a width dimension.

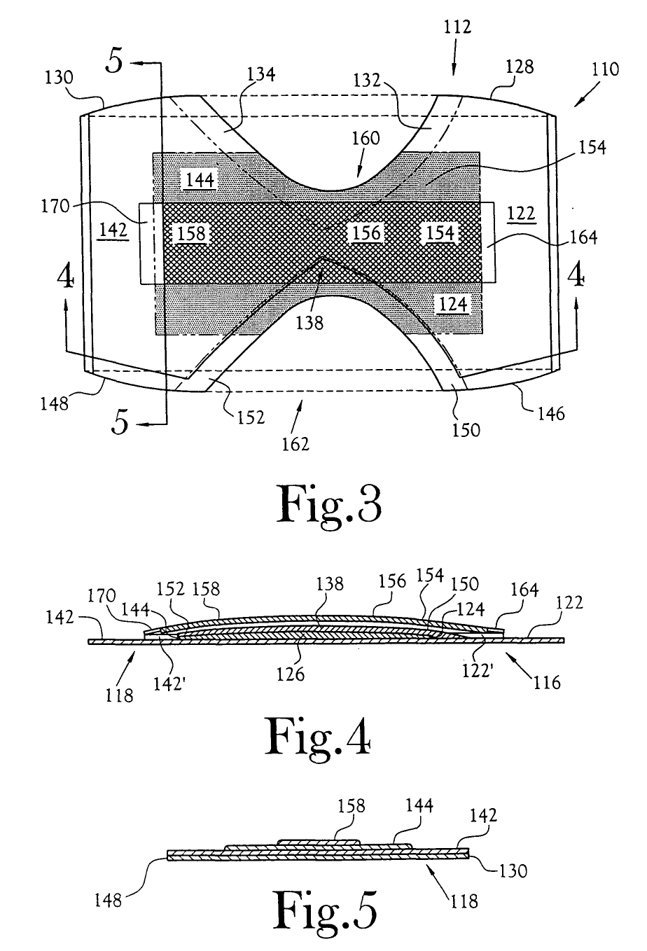

FIG. 3 illustrates an interior view of the panty 112 when the panty 112 is laid open by cutting along each generally continuous outer side 140. The front interior 122 of the panty 112 includes a generally continuous liner of absorbent material having an interior front liner 124, which extends through the crotch portion 117 to form an interior crotch liner 126, and extends to the rear interior 142 to form an interior rear liner 144. An additional absorbent crotch layer 138 is disposed interior of crotch liner 126, and is extended through the crotch portion 117 of the panty 112. The interior front liner 124 is integrally attached to an interior front 122 portion of the panty 112. The front liner 124 provides an absorbent layer positioned against the lower abdomen of the wearer during wear of the panty 112. The rear liner 144 is integrally attached to an interior rear 142 portion of the panty 112. The rear liner 144 provides an absorbent layer positioned to extend across the width of the buttocks of the wearer. Rear liner 144 extends to a position that is about at a mid-height of the rear portion 142 of the panty 112. The absorbent crotch layer 138 provides a continuous connection between the front liner 124 and the rear liner 144 without a seam to minimize leakage of excreted liquids therethrough. The absorbent materials of each portion of the liner 124, 138, 144 are composed of a mix of absorbent materials that typically provides a plurality of types of absorbing material including cellulose based and non-cellulose based absorbing materials. An alternate embodiment provides a triple layer of absorbent liner materials along the crotch portion 117 of the panty 112, including an exterior absorbent layer 126 disposed to extend through the crotch portion 117, the crotch absorbent layer 138 disposed interior of layer 126, and an interior faced portion 156 of removable layer 154, 156, 158 as illustrated in FIGS. 3 and 4. The interior faced removable layer 154, 156, 158 is discussed below.

At least one of the outer sides 120, 140 includes a seam (not shown) that is not visible from casual inspection of the outer surfaces, but is visible from inspection of the interior surface of either side 120, 140. The left inner portion of the outer side 120 is formed by attachment of a first front, interior side portion 128 of material to a second rear, interior side portion 130 of material by an attaching means known to those skilled in the art. The right inner portion of the outer side 140 is formed by attachment of a third front, interior side portion 146 of material to a fourth rear, interior side portion 148 of material by an attaching means. Either of the outer sides 120, 140 are detachable by the wearer tearing along a seam for ease of removal after use and soiling of the panty 112.

As illustrated in FIGS. 3 and 4, each leg opening 160, 162 includes an interior border having absorbent material extended along each interior edge portion of each leg opening 160, 162. The generally continuous interior borders of each leg opening include absorbent strips 132, 134 for the left leg opening 160, and absorbent strips 150, 152 for the right leg opening 162. Each absorbent strip is composed of a liquid absorbing material commonly utilized in disposable diapers such as cellulose-based liner materials, comminuted wood pulp fibers, and/or absorbent polymers that are in a form of wadding or an absorbent foam layer that typically provides at least one layer of absorbable material on the interior border of each respective leg opening 160, 162. The absorbent strips 132, 134, 150, 152 include at least a single layer of absorbable material for collection of leakage of excreted fluids that escape from the interior portions of the liner 124, 138, 144. An alternative embodiment includes at least two layers of absorbable materials (not shown) for each absorbent strip 132, 134, 150, 152. An alternative embodiment includes elasticized material provided proximate with each absorbent strip 132, 134, 150, 152, to provide expandable and contractable leg openings.

In one embodiment, the undergarment 110 includes an interior faced front portion 122 having at least one strip 122′ of tacky, adherent material that is disposed laterally along the interior faced front portion 122 at about a middle height of the front portion 122. The interior faced rear portion 142 includes at least one strip 142′ of tacky, adherent material that is disposed laterally along the interior faced rear portion 142 at about a middle height of the rear portion 142. An additional layer of protection is provided by providing a removable layer 154, 156, 158 that is a substantially continuous layer of absorbent materials is removably positioned onto, and interior of, the front liner 124, crotch liner 138, and rear liner 144. The removable layer 154, 156, 158 is illustrated in FIGS. 3, 4 and 5, and includes a front absorbent layer 154 having a front edge tab 164 that is tacky on a side faced toward front liner 124. The front edge tab 164 is intended to be positioned against the at least one strip 122′ of tacky, adherent material. The removable layer further includes a crotch absorbent layer 156 that is positioned on an interior side of crotch liner 138. The removable layer 154, 156, 158 also includes a removable rear absorbent layer 158 having a rear edge tab 170 that is tacky on a side faced toward rear liner 144. The rear edge tab 170 is intended to be positioned against the at least one strip 142′ of tacky, adherent material. The removable layer 154, 156, 158 of absorbent materials is removable by the wearer after the removable layer becomes soiled, by lowering the undergarment during visitation to a rest room, separating front edge tab 164 from the front strip 122′ of adherent material, and separating rear edge tab 170 from rear strip 142′ of adherent material. Upon removing the removable layer 154, 156, 158, the wearer may continue wearing the undergarment while having a dry and clean layer of absorbing materials against the crotch area as provided by the absorbent materials of each portion of liner 124, 138, 144. The interior circumferential border of absorbent materials 132, 134, 150, 152 remain positioned along the interior faced surfaces of each of the two leg openings 160, 162. The removable layer 154, 156, 158 is disposable by the wearer, and a replacement removable layer 154, 156, 158 may be positioned by the wearer or the undergarment may be worn without the removable layer 154, 156, 158, with the wearer relying on the absorption protection provided by absorbent materials of liner 124, 138, 144, absorbent layer 126, and each border of absorbent materials 132, 134, 150, 152 that remain positioned along each interior faced surface of the two leg openings 160, 162.

The combination of features of the disposable menstrual undergarment provides protection from leaks of menstrual fluid for young females, and provides protection from leaks of excreted body fluids from incontinent children or adults needing special assistance, while providing an undergarment that is not bulky, is easily removable, and is inexpensive to allow for disposal after use without the need for cleaning and reuse.

While numerous embodiments are illustrated and described, it will be recognized that alternative embodiments of the disclosed invention may be employed without departing from the spirit and the scope of the invention as set forth in the appended claims. Further, the disclosed invention is intended to cover all modifications and alternate embodiments falling within the spirit and the scope of the invention as set forth in the appended claims.

Having thus described the aforementioned invention, we claim:

1. A disposable menstrual undergarment for absorption of body fluids excreted by a wearer, comprising:
a front interior portion attached to a rear interior portion along opposed side portions of an undergarment, said front and rear interior portions having an interior surface;
a crotch interior portion joining said front interior portion and said rear interior portion of said undergarment;
a waistband of generally continuous material;
two leg openings through said opposed side portions and adjacent said crotch portion, each of said two leg openings having an interior border;
an exterior layer of said undergarment, said exterior layer of breathable non-porous material;
a plurality of layers disposed on interior surfaces of said undergarment, including:
a middle layer of absorbent materials having a front layer, a crotch layer and a rear layer positioned on each of respective interior surfaces of said front portion, rear portion, and said crotch portion of said undergarment;
an interior faced layer of absorbent materials removably disposed on said middle layer, said interior faced layer extends from said interior surface of said front portion, across said crotch portion, and to said rear portion; and
at least one strip of absorbent materials attachable along said interior border of each of said two leg openings, said at least one strip of absorbent materials being generally continuous along each interior border of each of said two leg openings;
whereby said interior faced layer of absorbent materials is removable by the wearer if soiled with said middle layer and said at least one strip of absorbent materials along each interior border of said leg openings provide for additional absorption of body fluids excreted by the wearer of the undergarment after removal of said interior faced layer.

2. The disposable menstrual undergarment of claim 1 wherein said undergarment including said exterior layer and said waistband being composed of cellulose-based material, said cellulose-based material having a plurality of stretchable filaments therein, said waistband is stretchable in a width dimension, said interior surface of said front interior portion, said rear interior portion, and said crotch interior portion having a layer of absorbent materials thereon.

3. The disposable menstrual undergarment of claim 2 wherein said interior layer of absorbent materials and said at least one strip of absorbent materials along each interior border are composed of a cellulose-based material having at least one layer of generally continuous absorbent materials along interior surfaces of said front portion, said rear portion, said crotch portion, and each interior border of said undergarment.

4. The disposable menstrual undergarment of claim 2 wherein said interior faced layer of absorbent materials including:
a front layer removably disposed proximal on said front portion of said middle layer on said front portion of said undergarment;
a rear layer removably disposed proximal on said rear portion of said middle layer on said rear portion of said undergarment; and
a crotch layer connected between said front layer and said rear layer of said middle layer on said crotch portion of said middle layer of said undergarment.

5. The disposable menstrual undergarment of claim 4 including said front layer having a front tab having one side of adherence material thereon, said front tab extends from a leading edge of said front layer of said interior faced layer of absorbent materials, and further including said rear layer having a rear tab having one side of adherence material thereon, said rear tab extends from a trailing edge of said rear layer of said interior faced layer of absorbent materials, said front tab is releasably attachable against said front interior portion of said undergarment, said rear tab is releasably attachable against said rear interior portion of said undergarment.

6. The disposable menstrual undergarment of claim 1 wherein said middle layer of absorbent materials is integrally attached to said interior surface of said front portion, rear portion, and said crotch portion to provide a continuous interior layer of absorbent materials composed of an absorbent polymer layer.

7. The disposable menstrual undergarment of claim 3 wherein at least one strip of absorbent materials along each interior border of each of said leg openings is composed of a cellulose-based absorbent layer or an absorbent polymer layer.

8. A disposable menstrual undergarment for absorption of menstrual fluids excreted by a wearer, comprising:
an undergarment having an exterior layer of breathable, non-porous and stretchable material, said undergarment including an interior surface of said exterior layer, said interior surface including a front portion attached to a rear portion, a crotch portion joining said front portion to said rear portion, and two leg openings on opposed sides of said crotch portion;
an interior circumferential border of absorbent materials positioned along interior faced surfaces of each of said two leg openings; and
a middle layer of absorbent materials positioned on each of an interior surface of said front portion, rear portion, and said crotch portion, said middle layer extended laterally to overlap said interior circumferential border of absorbent materials positioned along said interior faced surfaces of each of said two leg openings;
an interior faced layer of absorbent materials removably disposed on said middle layer, said interior layer is positioned on said interior surface of said front portion of said middle layer, across said crotch portion, and on said rear portion of said middle layer, said interior faced layer is removably attached on said front portion and said rear portion of said middle layer of absorbent materials;
whereby said interior faced layer of absorbent materials is removable by the wearer from said middle layer when said interior faced layer is soiled, with said middle layer remaining fixed against said interior circumferential border of absorbent materials positioned along interior faced surfaces of each of said two leg openings provide for absorption of menstrual fluids excreted by the wearer of the undergarment.

9. The disposable menstrual undergarment of claim 8 wherein said interior faced layer further includes a front edge having at least two extension tabs each having one side of adherence material thereon, said at least two extension tabs extend from said front edge of said interior faced layer of absorbent materials for adhesive contact with said interior surface of said front portion of said undergarment, and further including said interior faced layer having a rear edge having at least two extension tabs each having one side of adherence material thereon, said at least two extension tabs extend from said rear edge of said interior faced layer of absorbent materials for adhesive contact with said interior surface of said rear portion of said undergarment.

10. The disposable menstrual undergarment of claim 9 wherein said undergarment including said exterior layer and said waistband being composed of cellulose-based material, said cellulose-based material having a plurality of stretchable filaments therein, said waistband is stretchable in a width dimension, said interior surface of said front portion, said rear portion, and said crotch portion having a layer of absorbent materials thereon.
